(19) United States
(12) Patent Application Publication
(10) Pub. No.: US 2017/0313970 A1
(43) Pub. Date: Nov. 2, 2017

(54) DEVICE FOR DETERMINING AND MONITORING THE PHYSIOLOGICAL STATES OF MICROBIAL CULTURES IN EACH INDIVIDUAL MICROBIOREACTOR OF A MICROTITER PLATE

(71) Applicant: Rheinisch-Westfälisch Technische Hochschule (RWTH) Aachen, Aachen (DE)

(72) Inventors: Jochen Büchs, Aachen (DE); David Flitsch, Aachen (DE); Georg Wandrey, Aachen (DE); Martin Kunze, Rottenburg (DE); Heiner Giese, Solingen (DE)

(21) Appl. No.: 15/498,997

(22) Filed: Apr. 27, 2017

(30) Foreign Application Priority Data
Apr. 27, 2016 (EP) 16167322.3

(51) Int. Cl. C12M 1/34

(52) U.S. Cl. CPC C12M 41/46

(57) ABSTRACT

A device for determining and monitoring the physiological state of microbial cultures in each individual microbioreactor of a microtiter plate, wherein a gas space of each microbioreactor of the microtiter plate is accessible via an inlet opening and outlet opening, includes means for shaking the microtiter plate and a gas supply system suitable for purging the gas space of each microbioreactor with a stream of purge gas in a purging phase. A shut-off device is arranged directly on each microbioreactor for interrupting the stream of purge gas. The flow resistances in the gas supply system and the flow resistance of each microbioreactor are configured so that the stream of purge gas in the purging phase is substantially equal in all of the microbioreactors. The device includes a measuring device configured to detect the physiological state of the microbial culture in each individual microbioreactor.

### CROSS-REFERENCE TO RELATED APPLICATIONS

The present application claims priority under 35 U.S.C. §119(e) to European Patent Application EP 16 167 322, filed on Apr. 27, 2016, the entire contents of which are incorporated herein by reference.

### BACKGROUND OF THE INVENTION

The invention relates to a device for determining and monitoring the physiological state of microbial cultures in each individual microbioreactor of a microtiter plate.

DE 44 15 444 C2 discloses a method and a device for determining and monitoring the physiological state of microbial cultures by quasi continuous measurement of the respiration activities in a shaken measuring flask under sterile conditions, wherein the measuring flask in a purge phase for supplying the microbial cultures with purge gas, and then in a measuring phase the stream of purge gas into the gas space of the measuring flask is interrupted by at least one shut-off means, and at least one parameter representative of the respiration activity is detected by at least one measuring device and, after conversion to an electrical signal, the parameter is processed in a control computer. In order to monitor the time profile of cultures of aerobic microorganisms in the measuring flasks, the oxygen transfer rate (OTR) is detected quasi continuously under sterile conditions. In the measuring phase, the gas space in the measuring flask is shut off via the at least one controllable shut-off means and the decline of the oxygen partial pressure is measured using a sterilizable pO₂ electrode. Failure of this pO₂ electrode forces the discontinuation of the test, since it cannot be changed without contaminating the culture. Moreover, the method known from DE 44 15 444 C2 requires the use of sterilizable transducers.

EP 0 905 229 B1 therefore proposes detecting each parameter by using a sterile separating means between the gas space of the measuring flask and the transducer. To replace the transducer, the latter is released from the measuring flask, while the separating means remains on the measuring flask.

The prior art also discloses microtiter plates as high throughput systems for cultivation of microbial cultures. In recent times, microtiter plates have supplanted the classical shaker flask as a culture system. By virtue of the constant miniaturization, many cultivations can be carried out in parallel. The sample volumes examined in the microtiter plates are smaller, which saves on resources and cuts costs.

### BRIEF SUMMARY OF THE INVENTION

An object of the invention is to provide a device for determining and monitoring the physiological state of microbial cultures in the microbioreactors (wells) of a microtiter plate, where the cultivation in the individual microbioreactors can be carried out under substantially equal test conditions.

To create substantially equal test conditions in each microbioreactor of the microtiter plate, it is necessary to keep the dead volume in the gas delivery of the purge gas as low as possible and to supply all the microbioreactors of the microtiter plate with the same quantity of purge gas. To reduce the dead volume, the shut-off means for interrupting the stream of purge gas are arranged directly on each microbioreactor. This has the effect that, after shut-off in the measuring phase, there is no further, uncontrolled admission of purge gas into the microbioreactors. Moreover, the flow resistances in the gas supply system and the flow resistance of each microbioreactor are configured in such a way that the stream of purge gas in the purging phase is substantially equal in all of the microbioreactors. The term substantially equal means that minor differences such as those due to manufacturing tolerances and/or differences in the length of the lines to the microbioreactors that do not affect the results are encompassed. To achieve this goal, the flow resistance of each microbioreactor is so high that the flow resistances of the gas supply system, namely the flow resistance of the delivery and removal lines for purge gas leading to the individual microbioreactors, are negligible.

The flow resistance of each microbioreactor is at least a factor of 50, preferably at least a factor of 100, higher than the flow resistances in the gas supply system. By virtue of this configuration of the flow resistances, there is no need for an individual device controlling the stream of purge gas for each microbioreactor in order to ensure a substantially equal stream of purge gas in all of the microbioreactors.

In the measuring phase which follows the purging phase, as in the quasi continuous measurement of the respiration activities in the shaken measuring flask, the physiological state of the microbial culture is detected with the aid of measuring device in each individual microbioreactor.

The gas supply system of the device according to the invention comprises a purge gas feed-in and a gas distribution system. As purge gas feed-in, it is possible, for example, to consider at least one reservoir with purge gas or at least one network that conducts purge gas.

If the feed-in comprises several reservoirs or several networks that provide different purge gases, a gas mixer unit can optionally be arranged between the purge gas feed-in and the gas distribution system. With the mixer unit, one of the purge gases or a mixture of different purge gases can be deliberately fed into the gas distribution system.

The gas distribution system for the purge gas preferably has a central delivery line, which extends from the gas feed-in to a subdistribution arranged on the microtiter plate for the purge gas. The subdistribution has delivery lines or channels for leading the purge gas to the individual microbioreactors, and also removal lines or removal channels for leading the purge gas out of the microbioreactors.

Possible purge gases are in particular air, oxygen and carbon dioxide, preferably mixtures of the aforementioned gases.

The provision of a central delivery line for the purge gas considerably reduces the layout in terms of equipment for the gas supply system. In particular, a single flow-controlling component, for example a pump or a flow regulator, can be arranged in the central delivery line in order to regulate the stream of purge gas, which is distributed to the microbioreactors. The high and substantially equal flow resistance of each microbioreactor has the effect that the stream of purge gas within the subdistribution is divided into substantially equal sub-streams and each individual microbioreactor is supplied identically with purge gas. By virtue of the substantially equal and much higher flow resistances of all the microbioreactors by comparison with the flow resistances in the delivery and removal lines to the individual microbioreactors, these lines do not need to have an identical length and/or an identical cross section, since the different flow resistances of the lines are negligible.

The central delivery line in the gas supply system additionally affords the possibility of providing a wash bottle for humidifying the purge gas centrally for all the microbioreactors. It corresponds to the physical circumstances that fermentation solutions lose moisture when they come into contact with dry purge gas. This loss of moisture can be compensated by humidifying the purge gases in wash bottles.

The shut-off means arranged on each microbioreactor has at least one inlet valve which comprises a valve seat, surrounding the inlet opening, and also a pneumatically actuated shut-off membrane for opening and closing the inlet opening. Preferably, however, the stream of purge gas in each microbioreactor is controlled by inlet and outlet valves. The pneumatically actuated shut-off membrane contributes to a compact structure of the valves, allowing the shut-off means to be arranged directly on each microbioreactor despite the small amount of available space. In addition, the dead volume is further reduced by virtue of the membrane principle of the valves used. By virtue of the material and the material quality of the shut-off membrane, it is moreover possible to reduce deposits on the closure body of the valve and thus to reduce the risk of contamination of the microbial culture.

The inlet valves of a plurality of microbioreactors can be actuated synchronously if a pressure chamber which can be acted upon by underpressure and/or overpressure, and which is configured for simultaneous pneumatic actuation of the shut-off membrane of several inlet valves, is arranged on the side of the shut-off membrane of several inlet valves that faces away from the valve seat. In particular, a wall surface of the pressure chamber is formed by the shut-off membrane which, as a result of being acted upon by underpressure or overpressure, either moves into the interior of the pressure chamber or moves outward. The membrane-controlled outlet valves of several microbioreactors can in the same way be actuated synchronously with the aid of a pressure chamber which can be acted upon by underpressure and/or overpressure.

The inlet valves and the outlet valves of the device are preferably controlled with a time delay. At the start of the measuring phase, all of the inlet valves are at first synchronously closed. With a time delay, for example of one second, all of the outlet valves are then closed synchronously. This staggered closure of all the inlet and outlet valves ensures that a pressure compensation between the gas space of each microbioreactor and the surrounding atmosphere has in each case taken place before the gas space is definitively closed. This results in a clearly defined reference pressure within the gas space during the measuring phase. At the end of the measuring phase, all of the outlet valves are at first synchronously opened. With a time delay, for example of one second, all of the inlet valves are then opened synchronously.

For design reasons, and in the interest of a substantially equal supply of purge gas to all the microbioreactors, the shut-off means of all the microbioreactors are exactly identical in number, type, size and shape. The high flow resistance of each microbioreactor with respect to the gas supply system is preferably afforded by the inlet opening of each microbioreactor surrounded by the valve seat. The integration of the flow resistance in the valve seat or the inlet opening takes account of the confined spatial conditions. In particular, no installation space is needed in the gas distribution system upstream from the shut-off means in order to provide a separate flow resistance there, for example in the form of a throttle.

To avoid a backing-up pressure within the microbioreactors during the purging phase, provision is made, in an advantageous embodiment of the invention, that the cross section of the inlet opening of each microbioreactor is smaller than the cross section of the outlet opening of each microbioreactor. The cross section of each outlet opening is preferably at least five times as great as the cross section of each inlet opening.

The measuring device are configured for detecting at least one parameter of the microbial culture representative of the respiration activity in each microbioreactor. In a preferred embodiment, the changes in partial pressure of oxygen and carbon dioxide over time are detected on the basis of the respiration activity. In a measurement computer, the decline of the oxygen partial pressure is converted to the OTR, and the decline of the carbon dioxide partial pressure is converted to the CTR. In a simplified embodiment of the invention, only the decline of the oxygen partial pressure is detected and is converted to the OTR in the measurement computer.

The device according to the invention preferably uses microtiter plates with 16 to 384 microbioreactors. To be able to detect the physiological state of the microbial culture despite the confined spatial conditions in each microbioreactor, passive measuring device are preferably used which comprise at least one passive measuring element which is arranged in each microbioreactor, and of which the measurement signal changes as a result of a change of the respiration activity.

The transducer for converting the measurement signal to electrical signals is not designed integrally with the passive measuring element and does not then have to be arranged on the microtiter plate, and instead it can be arranged at a distance from the shaker for the microtiter plate. With the aid of transmission lines, the measurement signals are transmitted between each passive measuring element and one of the transducers.

With a passive measuring device of this kind, in order to permit contactless detection of the respiration activity within each microbioreactor, each passive measuring element is preferably designed as an indicator layer which is arranged permanently on a transparent surface of the microbioreactor and which reacts to changes of the gas concentration in the gas interior by changing the emitted electromagnetic radiation in the optical range. The electromagnetic radiation (measurement signal) is coupled through the surface of the microbioreactor, transparent to electromagnetic radiation, into an optical fiber as transmission line and is transmitted to the transducer.

In a preferred embodiment, the detection of the parameter representative of the respiration activity is based on measuring a decrease in the fluorescence lifetime of a fluorescent dye in the presence of the corresponding analyte. The physical mechanism is known as “quenching” and is described by the Stern-Vollmer equation.

In order to avoid elaborate time-resolved measurements of the fluorescence lifetime, the indicator layer in a preferred embodiment of the invention is irradiated by an intensity-modulated light source from the transducer. This excitation light is modulated with a fixed frequency and is transmitted by the optical fiber from the transducer to the indicator layer. The intensity-modulated excitation of the fluorescent dye has the effect that the resulting fluorescent light is detected with the same frequency by the transducer with a time delay. This time delay is directly proportional to the lifetime of the fluorescence and is thus dependent on the partial pressure of the corresponding analyte. The detection takes place in the transducer by a photodiode as optoelectronic sensor with an upstream optical filter adapted to the fluorescent light. This prevents surrounding light of another wavelength, or the excitation light reflected back on boundary faces, from impinging on the photodiode. To ensure that surrounding light, which can pass through the optical filter on account of a similar wavelength to the fluorescent light, is not incorrectly accepted as fluorescent light, a so-called lock-in amplifier is integrated in each transducer.

This evaluates the electrical signals which arise from the light incident on the photodiode, such that all the signals with a frequency deviating from the modulated light intensity of the transducer are not taken into account.

For this reason, the measurement is not influenced by spectrally similar surrounding light as long as the intensity of this surrounding light is not modulated with the same frequency as that of the excitation light.

In order to realize the highest possible data density in the detection of the measurement parameters that are representative of the respiration activity, a parallel measurement detection is preferable to a sequential measurement detection. To ensure that the transducers cannot influence each other as a result of unwanted fluorescent light capture from nearby indicator layers, the corresponding modulation frequencies of the modulated light sources of the transducers should differ, so that it is possible for the lock-in amplifiers of the transducers to ignore this light likewise in the evaluation of the corresponding electrical signals of the photodiode.

If the standard frequency with which the intensity of the excitation light is modulated is 4,000 Hz for example, a difference of about 100 Hz is sufficient, for the excitation light which is introduced into mutually adjacent microbioreactors, in order to suppress mutual influencing of the transducers.

In order to obtain a maximum data density in the measuring phase and thus increase the measuring accuracy, each passive measuring element is preferably fixedly linked to its assigned transducer via a transmission line. This signifies that the measuring device has an identical number of passive measuring elements and transducers. If the transducers are optoelectronic components for converting the measurement signals to electrical signals, this entails not inconsiderable costs. With the aid of an optical multiplexer, a compromise is possible between the required data density and the number of transducers. The optical fibers are connected to the inlets of the optical multiplexer and the transducers are connected to the outlets thereof. The measurement signals at different inlets can be switched through in succession to one of the outlets with the aid of the multiplexer. As a result, the passive measuring elements of several microbioreactors share one transducer with the aid of the multiplexer.

To supply purge gas to the microbioreactors in the confined installation space of the microtiter plate, in an advantageous embodiment of the invention all of the shut-off means and the subdistribution for the purge gas are integrated in a cover which can be fitted in the manner of a lid onto the microtiter plate.

If the shut-off means are designed as pneumatically actuated valves with a shut-off membrane, the pressure chambers which can be acted upon by underpressure and/or overpressure, and which are provided for actuation of the shut-off membrane of the valves, are integrated in the cover.

A sterile barrier is preferably arranged between the microtiter plate and the cover and extends across the whole surface of the microtiter plate. The sterile barrier is gas-permeable and at the same time prevents contamination of the microbioreactors with foreign organisms. Since the shut-off means are all located in the cover, i.e., on one and the same side of the sterile barrier, only one sterile barrier per microbioreactor is needed, in contrast to the conventional shake flask technique. In the conventional shake flask technique, sterile barriers are arranged in the inlet and outlet and upstream of the measuring device.

Owing to the type of construction, in the microtiter plate according to the invention the inlet and outlet openings are arranged relatively close to each other on the top of each microbioreactor. Therefore, a barrier is preferably arranged between the inlet opening and outlet opening of each microbioreactor in such a way that a short circuit of the stream of purge gas between inlet and outlet is suppressed.

The manufacture of the cover with integrated functional elements can be simplified if the cover is composed of a plurality of plates that are interconnected to form a stack.

### BRIEF DESCRIPTION OF THE DRAWINGS

The invention is explained in more detail below with reference to the figures, in which:

FIG. 1 shows a schematic view of a device according to the invention,

FIG. 2 shows a schematic sectional view through a microbioreactor of the device according to the invention,

FIGS. 3a and 3b show schematic views of pneumatically actuated valves of a microbioreactor according to FIG. 2, in different switching positions,

FIGS. 4a and 4b show schematic sectional views along the lines 33, 34 according to FIG. 2,

FIG. 5 shows a schematic sectional view of a cover for a microtiter plate according to the present invention,

FIG. 6a shows an isometric view of an optical multiplexer, and

FIG. 6b shows a schematic plan view of the multiplexer in order to illustrate the mode of operation.

### DETAILED DESCRIPTION OF THE PREFERRED EMBODIMENTS

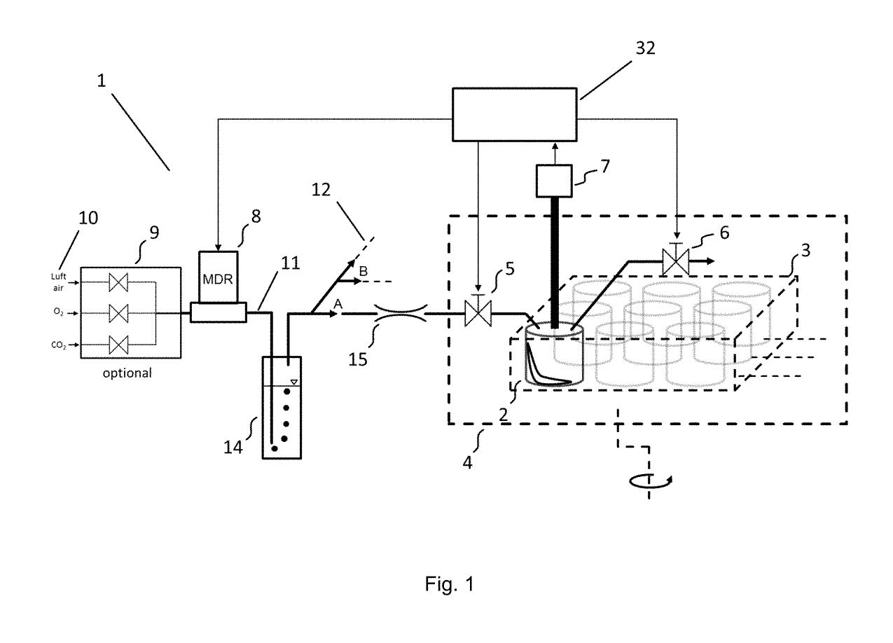

FIG. 1 shows a device for determining the physiological state of microbial cultures in each individual microbioreactor (2) of a microtiter plate (3), wherein the microtiter plate (3) is set in a shaking motion by a shaker device including a shaker tray (4). A controlled, uniform supply of a purge gas to each individual microbioreactor (2) is intended to be permitted according to the invention under the shaken conditions. With a device according to the invention, it is possible, for example, to detect an oxygen limitation in each individual microbioreactor (2), without directly measuring the dissolved oxygen concentration in the microbial culture.

The oxygen transfer rate (OTR) is calculated as follows:

$$O T R = \frac{{d}_{PO 2}}{dt} \cdot \frac{Vg}{{V}_{f 1} \cdot R \cdot T} \qquad formula 1$$

dpO₂/dt: differential quotient for oxygen particle pressure over time [bar/min]
V_{g}: gas volume of the microbioreactor [ml]
V_{fl}: liquid volume of the microbioreactor [ml]
T: temperature [K]
R: gas constant [bar*l/mol/K]

From the shape of the decay curve of the oxygen partial pressure in the measuring phase, it is possible to ascertain whether the oxygen transport is dependent on the oxygen consumption rate of the microorganisms (limited by reaction) or on the mass transfer (gas phase to liquid phase) (limited by mass transfer). In the first case, the oxygen consumption is independent of the driving partial pressure gradient, i.e., the differential quotient from the formula 1 can be replaced by a difference quotient:

$$\frac{{Δ}_{PO 2}}{Δ t} = O T R \cdot \frac{{V}_{fl} \cdot R \cdot T}{Vg} \qquad formula 2$$

Formula 2 shows that, with a linear oxygen partial pressure drop in the measuring phase, there is no oxygen limitation of the culture.

If there is an oxygen limitation (limited by mass transfer), the oxygen consumption is no longer independent of the driving partial pressure gradient, and the equation for the partial pressure drop in the measuring phase is as follows:

$$\frac{ln \frac{{P}_{O {2}_{2}}}{{P}_{O {2}_{1}}}}{Δ t} = {k}_{L} a \cdot \frac{{V}_{fl} \cdot R \cdot T}{Vg \cdot He} \qquad formula 3$$

k_{L}a: volumetric mass transfer coefficient [l/h]
He: Henry's constant [bar*l/mol]
P_{O21}: oxygen partial pressure at the start of the measuring phase [bar]
P_{O22}: oxygen partial pressure at the end of the measuring phase [bar]

This dependency on the driving partial pressure gradient leads to a non-linear curve shape.

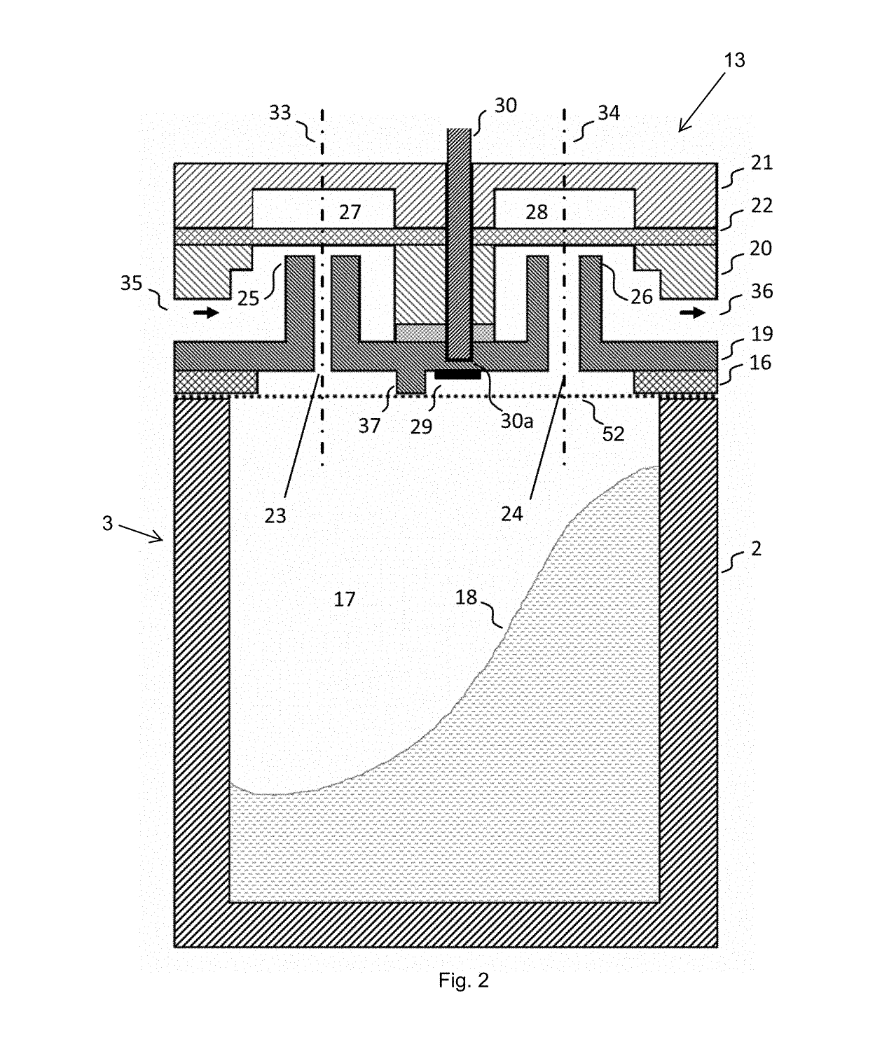

Each microbioreactor (2) has an inlet valve (5) and an outlet valve (6), which are closed in the measuring phase of the device. Both the inlet valve (5) and the outlet valve (6) are arranged directly on each microbioreactor, as can be seen from FIGS. 2-5. With the aid of measuring device (7), changes of the partial pressure of the purge gas are detected in each individual microbioreactor, and the transfer rates are calculated from these in a measurement computer (32). The measurement computer (32) moreover controls the inlet and outlet valves (5, 6), a mass flow regulator (8) and, if appropriate, an optional gas mixer unit (9).

With a feed-in (10), the purge gas is delivered to the individual microbioreactors (2) via a gas distribution system comprising a central delivery line (11) and a subdistribution (12), which for reasons of clarity is not shown in FIG. 1, and said purge gas is removed from them again via the outlet valves (6). The subdistribution (12) is arranged in a cover (13) which is fitted onto the microtiter plate (3) and which has further functional elements for controlling the purge gas.

A wash bottle (14) can additionally be arranged in the central delivery line (11) in order to compensate for loss of liquid caused by evaporation during cultivation. In order to ensure a substantially equal supply of purge gas in all of the microbioreactors (2), all of the inlet valves (5) and all of the outlet valves (6) of the microbioreactors (2) switch collectively in each case. Finally, the reference character (15) in FIG. 1 schematically indicates the flow resistance of each microbioreactor (2).

The structure of the cover (13) is explained in more detail below with reference to the partial view in FIG. 2 in conjunction with FIG. 1. To illustrate the integration of the inlet and outlet valves (5, 6) and their pneumatic actuation, the view is not to scale. Between the cover (13) and the microtiter plate (3), a seal (16) is provided in order to prevent an escape of gas from the gas space (17) above the microbial culture (18) in the closed state of the two valves (5, 6). In structural terms, the cover (13) is composed of a transparent bottom plate (19), a middle plate (20) and a top plate (21). A switching membrane (22) is arranged between the top plate (21) and the middle plate (20).

Inlet openings (23) and outlet openings (24) are located in the bottom plate (19), wherein the gas space (17) of each microbioreactor (2) is accessible via the inlet opening (23) and the outlet opening (24). Furthermore, the bottom plate (19) comprises a valve seat (25), which surrounds each inlet opening (23), and also a valve seat (26), which surrounds each outlet opening (24). The switching membrane (22) arranged between the middle plate (20) and the top plate (21) delimits first pressure chambers (27) and second pressure chambers (28). Depending on the controllable pressure prevailing inside the pressure chambers (27, 28), the elastic switching membrane (22) bears on the valve seats (25, 26) and closes the inlet and outlet valves (5, 6) collectively. The valve seats (25, 26) integrated in the bottom plate (19) form, together with the switching membrane (22) likewise integrated in the cover (13), all the shut-off means for interrupting the stream of purge gas into the microbioreactors (2).

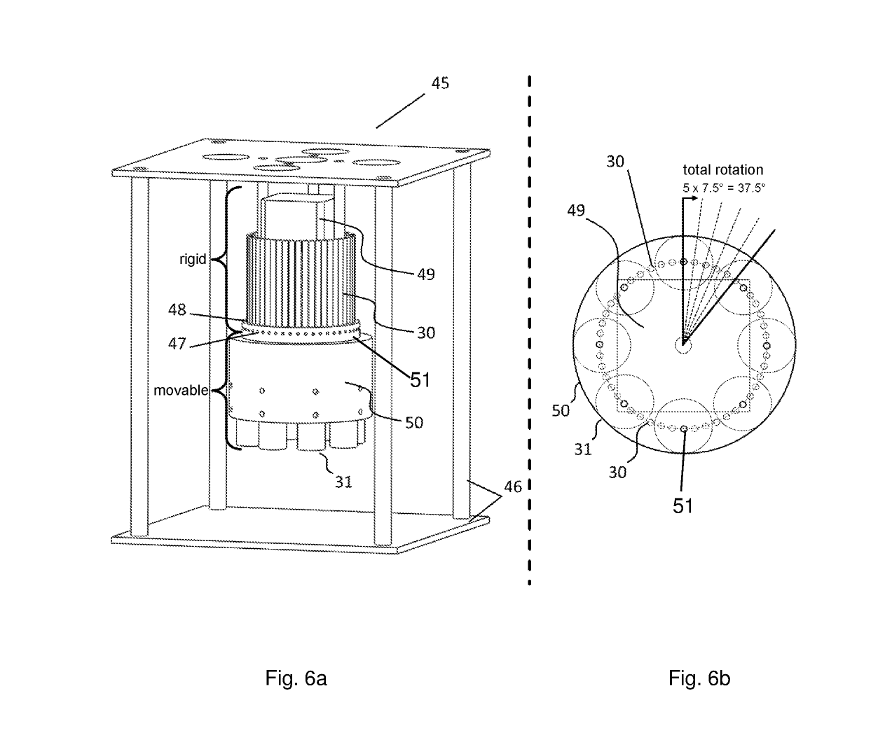

It will also be seen from FIG. 2 how the measuring device (7), which are shown merely schematically in FIG. 1, are partially integrated in the cover (13). A passive measuring element (29) is arranged on the surface of the transparent bottom plate (19) pointing toward the gas space (17) of each microbioreactor (2). The passive measuring element (29) is, for example, a fluorescence spot that is sensitive to partial pressure. The fluorescence spot reacts to changes of the gas concentration in the gas space (17) by changing the emitted electromagnetic radiation, which is transmitted contactlessly via a transmission line (30) from each microbioreactor (2) to a transducer (31) shown in FIGS. 6a and 6b. The transmission line (30), for example in the form of an optical fiber, is introduced, on the side lying opposite the passive measuring element (29), into a recess (30a) of the transparent cover (19).

The reference character (35) finally indicates a part of the line structure of the subdistribution (12) delivering the purge gas, and the reference character (36) indicates a part of the line structure of the subdistribution (12) carrying off the purge gas. In addition, a flow-conducting barrier (37) is arranged on the underside of the bottom plate (19) between the inlet opening (23) and the outlet opening (24) of each microbioreactor (2), which barrier prevents a short circuit flow of the purge gas directly between the inlet and outlet openings (23, 24) when the valves are closed.

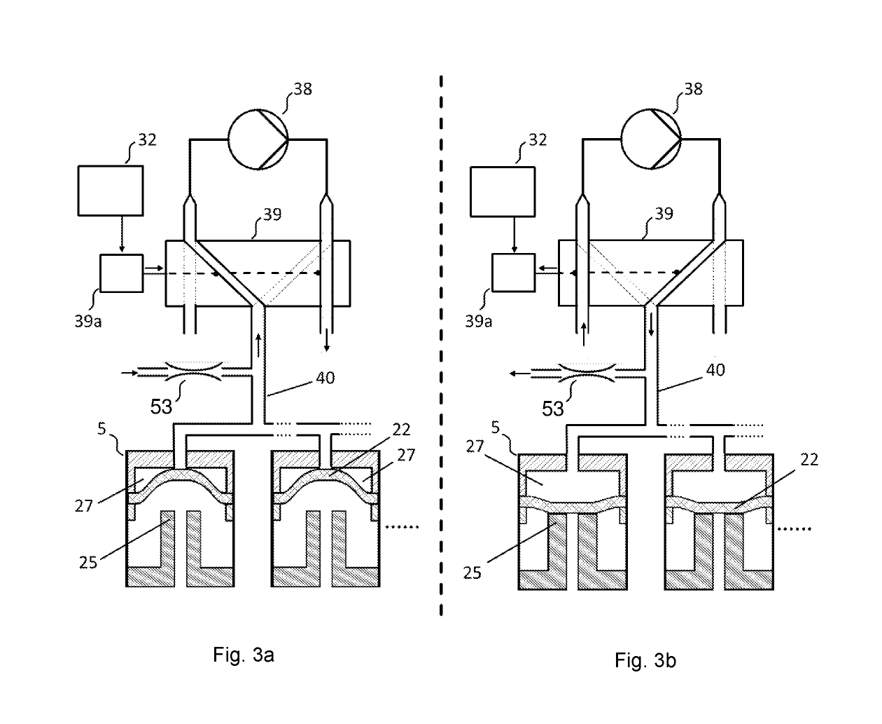

With reference to FIGS. 3a and 3b, the actuation of the inlet valves (5), controlled by the measurement computer (32), is explained in more detail below on the basis of a sectional view of the cover (13). To generate an underpressure or overpressure in a first pressure chamber (27), an electrically driven pump (38) is provided whose suction side and pressure side are connected to the ports of a 5/2-way valve (39). In the switching position of the 5/2-way valve (39) shown in FIG. 3a, the suction side of the pump (38) is connected to the first pressure chamber (27) via a line (40). The elastic switching membrane (22) is pulled in the direction of the upper face of the first pressure chamber (27) by the underpressure. During the opened state of the inlet valves (5), the underpressure is maintained in order to ensure a controlled switching position. In the switching position of the 5/2-way valve (39) shown in FIG. 3b, the first pressure chamber (27) is connected fluidically to the pressure side of the pump (38). The overpressure now prevailing in the first pressure chamber (27) now presses the elastic switching membrane (22) onto the valve seat (25) of each inlet valve (5).

Attached to the line (40) is a branch line with a throttle (53), which limits the underpressure in the switching state of the inlet valve (5) according to FIG. 3a and limits the overpressure in the switching state of the inlet valve (5) according to FIG. 3b.

In order to switch the 5/2-way valve (39), the latter is connected to the measurement computer (32) via an actuator (39a). The actuation of the outlet valves (6) with the aid of the 2nd pressure chamber (28) takes place in the same way, and therefore a separate explanation of the valve actuation is unnecessary.

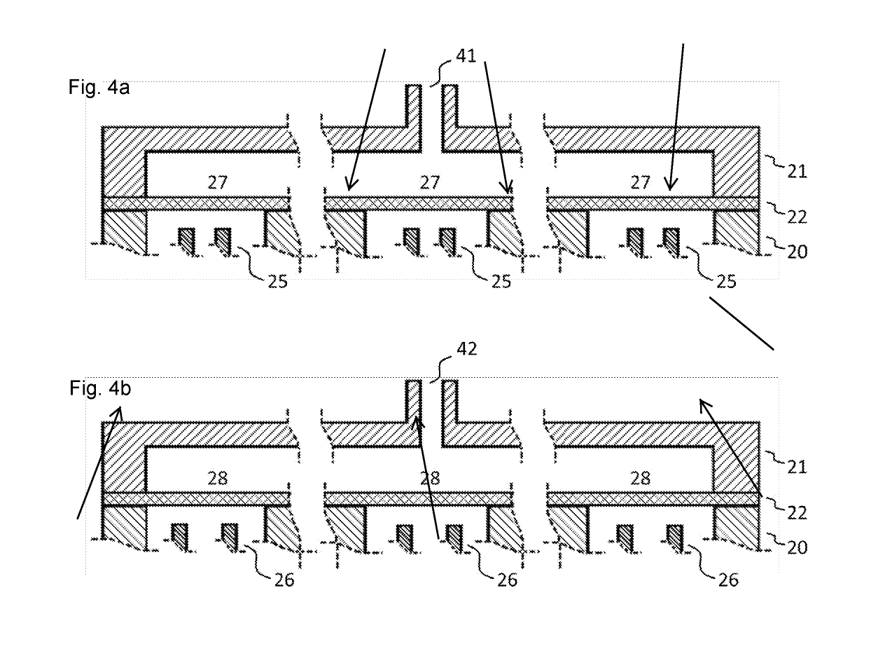

The cross sections shown in FIGS. 4a and 4b, taken along the section lines 33, 34 according to FIG. 2, illustrate how several inlet valves (5) arranged in a row are actuated via a common first pressure chamber (27) and how several outlet valves (6) arranged in a row are actuated via a common second pressure chamber (28). For this purpose, each first pressure chamber (27) and each second pressure chamber (28), for actuation of a row of inlet and outlet valves (5, 6), is connected via a port (41) and (42), respectively, to the valve control system shown in FIG. 3.

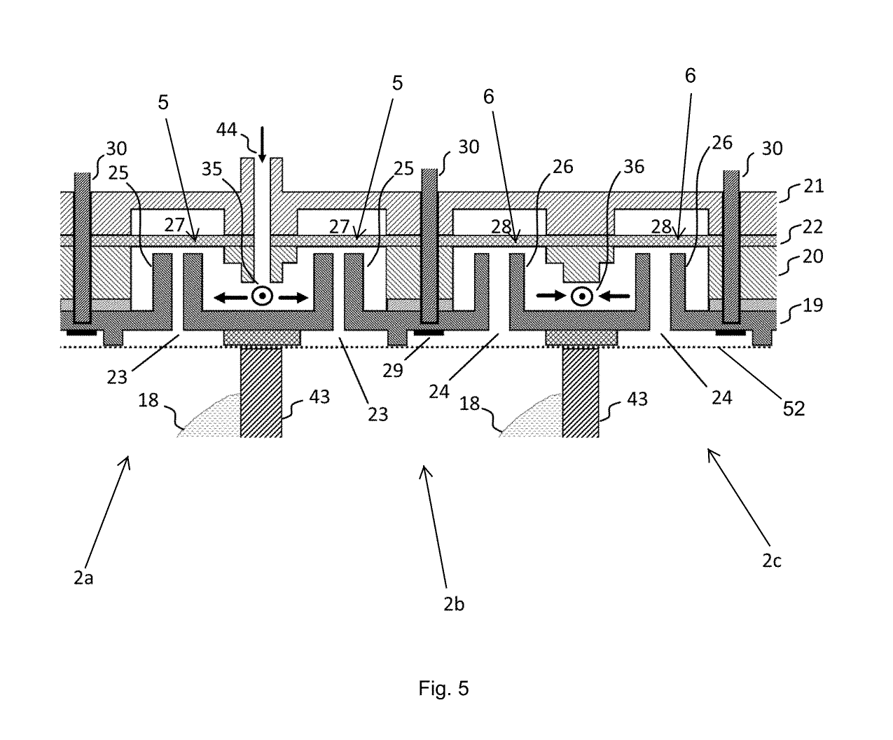

The partial section according to FIG. 5, corresponding to FIG. 2 in terms of the direction of sectioning, shows a total of three microbioreactors (2a, 2b, 2c) in order to illustrate the structure of the subdistribution (12) for the purge gas inside the cover (13). Each microbioreactor (2a, 2b, 2c) is a component part of a series of microbioreactors which extend perpendicularly with respect to the image plane and which are all separated from each other by partition walls (43). It will clearly be seen how the inlet valves (5) of the adjacent rows with microbioreactors (2a, 2b) are supplied with purge gas via the delivery line structure (35) in the opened state of the valves. It will moreover be seen how the outlet valves (6) of the adjacent rows with microbioreactors (2b, 2c) are fluidically connected to the removal line structure (36) for withdrawing the purge gas from the microbioreactors.

The central delivery line (11), shown in FIG. 1, of the gas supply system is attached to the port (44) opening into the delivery line structure (35). By way of further line structures of the subdistribution (12) that are not visible in the sectional view according to FIG. 5, the purge gas delivered via the port (44) arrives at further rows (not shown in FIG. 5) of inlet valves (5).

The length of the delivery and removal line structure (35, 36) to each individual microbioreactor (2a, 2b, 2c) is different. A uniform supply of purge gas to all of the microbioreactors is ensured in any case, since the cumulative flow resistances in the gas supply system as far as the respective microbioreactor (2a, 2b, 2c), i.e. in the delivery line (11) and subdistribution (12), are negligible compared to the flow resistance in the respective microbioreactor (2a, 2b, 2c). As a result, a single flow-controlling component, for example the mass flow regulator (8) shown in FIG. 1, is sufficient for a uniform supply of purge gas to all of the microbioreactors.

The respiration activity of the microbial cultures leads to a change of the purge gas concentration in the closed gas space (17) of each microbioreactor (2) and is linked with a change of the electromagnetic radiation emitted by the fluorescence spot and coupled into the optical fiber. The optical fibers are routed from the microtiter plate (3) to an optical multiplexer (45), which is shown in FIG. 6a and which can be set up next to the shaker tray (4) shown in FIG. 1. The optical multiplexer (45) comprises a frame (46) with an annular body (47) which is mounted in a stationary position and on which a number of first ports (48) are arranged corresponding to the number of transmission lines (30) (optical fibers). Moreover, a rotary drive (49), for example in the form of a stepping motor, is secured on the frame (46), its output shaft being connected to a cylindrical mount (50). The axis of rotation of the mount (50) extends coaxially with respect to the axis of the annular body (47). Second ports (51) of the multiplexer are arranged on the mount (50) in such a way that they can be brought into communication with the first ports (48), by rotating the mount (50), for the measurement signals and the excitation light of the modulatable light source. One of the optoelectronic transducers (31) is attached to each second port (51). The number of the passive measuring elements (29) is greater than the number of the transducers (31) by an integral multiple.

By actuation of the rotary drive, the electromagnetic signals at the first ports (48) can be switched through in succession to one of the second ports (51) and in this way delivered to one of the transducers (31), which converts the electromagnetic radiation delivered by the individual microbioreactors into electrical signals. Furthermore, the excitation light generated in the transducers (31) at the second ports (51) is switched through to one of the first ports (48) and in this way delivered to one of the passive measuring elements (29).

### LIST OF REFERENCE SIGNS

| No. | Designation |
|---|---|
| 1 | device |
| 2 a, b, c | microbioreactors |
| 3 | microtiter plate |
| 4 | shaker tray |
| 5 | inlet valve |
| 6 | outlet valve |
| 7 | measuring device |
| 8 | mass flow regulator |
| 9 | gas mixer unit |
| 10 | feed-in (purge gas) |
| 11 | central delivery line |
| 12 | subdistribution |
| 13 | cover |
| 14 | wash bottle |
| 15 | flow resistance |
| 16 | seal |
| 17 | gas space |
| 18 | microbial structure |
| 19 | bottom plate |
| 20 | middle plate |
| 21 | top plate |
| 22 | switching membrane |
| 23 | inlet opening |
| 24 | outlet opening |
| 25 | valve seat (inlet valve) |
| 26 | valve seat (outlet valve) |
| 27 | 1^{st} pressure chamber |
| 28 | 2^{nd} pressure chamber |
| 29 | passive measuring element |
| 30 | transmission line |
| 30a | recess |
| 31 | transducer |
| 32 | measurement computer |
| 33 | section line |
| 34 | section line |
| 35 | delivery line structure |
| 36 | removal line structure |
| 37 | barrier |
| 38 | pump |
| 39 | 5/2-way actuator |
| 39a | actuator |
| 40 | port |
| 41 | port of 1^{st} pressure chamber |
| 42 | port of 2^{nd} pressure chamber |
| 43 | partition walls |
| 44 | port |
| 45 | optical multiplexer |
| 46 | frame |
| 47 | annular body |
| 48 | first port |
| 49 | rotary drive |
| 50 | holder |
| 51 | second port |
| 52 | sterile barrier |
| 53 | throttle |

What is claimed is:

1. A device for determining and monitoring a physiological state of microbial cultures in each individual microbioreactor of a microtiter plate, comprising:
a shaker device for shaking the microtiter plate,
a gas supply system suitable for purging a gas space of each microbioreactor with a stream of purge gas through respective inlet openings and outlet openings for the each microbioreactor in a purging phase,
a shut-off device arrangeable directly on each microbioreactor and suitable for interrupting the stream of purge gas,
wherein flow resistances in the gas supply system and the flow resistance to the each microbioreactor are configured in such a way that the stream of purge gas in the purging phase is substantially equal in all of the microbioreactors, and
a measuring device configured to detect the physiological state of the microbial culture in each individual microbioreactor in a measuring phase while the stream of purge gas is interrupted.

2. The device according to claim 1, wherein the gas supply system comprises a purge gas feed-in and a gas distribution system including the inlet openings and the outlet openings, the gas distribution system being configured to deliver the fed-in purge gas and to remove the purge gas from the each individual microbioreactor.

3. The device according to claim 2, wherein the gas distribution system further comprises a central delivery line, which extends from the gas feed-in to a subdistribution arranged on the microtiter plate for conducting the purge gas to and from the inlet openings and the outlet openings.

4. The device according to claim 3, wherein the gas supply system includes a flow-controlling component arranged in the central delivery line.

5. The device according to claim 3, wherein the gas supply system includes a wash bottle arranged in the central delivery line.

6. The device according to claim 1, wherein the shut-off device includes an inlet valve with a valve seat surrounding the inlet opening and a pneumatically actuated shut-off membrane for opening and closing the inlet opening.

7. The device according to claim 6, wherein the shut-off device further includes an outlet valve with a valve seat surrounding the outlet opening and also a pneumatically actuated shut-off membrane for opening and closing the outlet opening.

8. The device according to claim 6, wherein a pressure chamber which can be acted upon by underpressure and/or overpressure, and which is configured for simultaneous pneumatic actuation of the shut-off membrane of several inlet valves, is arranged on the side of the shut-off membrane of several inlet valves that faces away from the valve seat.

9. The device according to claim 7, wherein a pressure chamber which can be acted upon by underpressure and/or overpressure, and which is configured for simultaneous pneumatic actuation of the shut-off membrane of several outlet valves, is arranged on the side of the shut-off membrane of several outlet valves that faces away from the valve seat.

10. The device according to claim 1, wherein the shut-off devices of all the microbioreactors are identical.

11. The device according to claim 1, wherein the flow resistance of each microbioreactor in the purging phase is higher than the flow resistances of the gas distribution system as far as the respective microbioreactor by at least a factor of 50.

12. The device according to claim 11, wherein the flow resistances of all the microbioreactors are substantially equal.

13. The device according to claim 1, wherein the inlet opening determines the flow resistance of the each microbioreactor.

14. The device according to claim 1, wherein the outlet opening determines the flow resistance of the each microbioreactor.

15. The device according to claim 1, wherein a cross sectional area of the inlet opening of each microbioreactor is smaller than a cross sectional area of the outlet opening of each microbioreactor.

16. The device according to claim 1, wherein at least a portion of the measuring device for detecting at least one parameter of the microbial culture representative of the respiration activity is arranged in each microbioreactor.

17. The device according to claim 1, wherein the measuring device comprises:
at least one passive measuring element arranged in each microbioreactor, a measurement signal of the at least one passive measuring element changing as a result of a change of respiration activity,
transducers for converting the measurement signals to electrical signals, and
transmission lines for transmitting the measurement signal between each passive measuring element and one of the transducers.

18. The device according to claim 17, wherein each the at least one passive measuring element is an indicator layer arranged permanently on a transparent surface of the microbioreactor and reacts to changes of the gas concentration in the gas interior by changing the emitted electromagnetic radiation, and wherein each of the transducers is designed as an optoelectronic component.

19. The device according to claim 17, further comprising an optical multiplexer with first ports and second ports, wherein
the number of the passive measuring elements and the number of the transmission lines is greater than the number of the transducers by an integral multiple, and
the transmission lines are connected to the first ports of the optical multiplexer and the transducers are connected to the second ports of the optical multiplexer, wherein measurement signals lying at different first ports can be switched through in succession to one of the second ports.

20. The device according to claim 17, wherein each of the transducers comprises a modulatable light source and an optoelectronic sensor, and the light sources of all the transducers have different modulation frequencies.

21. The device according to claim 21, wherein all of the shut-off devices are integrated in a cover that can be fitted onto the microtiter plate.

22. The device according to claim 3, the subdistribution for the purge gas is integrated in a cover that can be fitted onto the microtiter plate.

23. The device according to claim 8, wherein the pressure chamber which can be acted upon by underpressure and/or overpressure, and which are provided for pneumatic actuation of the shut-off membrane, is integrated in a cover that can be fitted onto the microtiter plate.

24. The device according to claim 21, further comprising a sterile barrier arranged between the microtiter plate and the cover.

25. The device according to claim 1, further comprising a barrier arranged between the inlet opening and outlet opening of each microbioreactor in such a way that a short circuit of the stream of purge gas between inlet and outlet is suppressed.

26. The device according to claim 1, the shaking device for shaking the microtiter plate includes a shaker tray.

27. The device according to claim 21, wherein the cover is composed of a plurality of interconnected plates.
